(19) United States
(12) Patent Application Publication
(10) Pub. No.: US 2016/0120692 A1
(43) Pub. Date: May 5, 2016

(54) Self-Warming Hands Free Eye Compress

(71) Applicant: Vicki Min Chen, Lexington, MA (US)

(72) Inventor: Vicki Min Chen, Lexington, MA (US)

(21) Appl. No.: 14/530,855

(22) Filed: Nov. 3, 2014

(51) Int. Cl. A61F 7/03

(52) U.S. Cl. CPC A61F 7/03; A61F 2007/0004

(57) ABSTRACT

Warm compresses are the most widely prescribed effective medical therapy for several common eye conditions namely dry eye syndrome, blepharitis and chalazia. These problems are caused by blockage of natural oil flow through pores located behind the eyelashes (meibomian glands). Warm compresses open these pores and restores normal flow of natural oils to the eye surface; these oils moisturize the eye by preventing evaporation of tears from the eye surface. The novel aspects of this invention that make it unique include the ability to generate long lasting, self-perpetuating moist heat, with hands free convenience. By allowing patients to continue to work, play, read or watch television while receiving the therapeutic benefits of a moist warm compress this invention is highly likely to increase compliance and improve quality of life for millions of patients with these common and highly troublesome eye conditions.

### TITLE OF INVENTION

Self-warming hands free eye compress, designed by Dr. Vicki M. Chen, Lexington, Mass., USA.

### CROSS-REFERENCE TO RELATED APPLICATIONS

N/A

### STATEMENT REGARDING FEDERALLY SPONSORED RESEARCH OR DEVELOPMENT

N/A

### REFERENCE TO SEQUENCE LISTING, A TABLE, OR A COMPUTER PROGRAM LISTING COMPACT DISC APPENDIX

N/A

### BACKGROUND OF THE INVENTION

### The Diseases

Warm compresses are the most widely prescribed effective medical therapy for several common eye conditions namely dry eye syndrome, blepharitis, and chalazia (common term: styes). Dry eye syndrome typically causes symptoms of dryness, pain while reading, driving or watching television, foreign body sensation, light sensitivity, and intermittent blurred vision. Blepharitis causes similar symptoms, but pain and foreign body sensation are often more severe than seen in dry eye syndrome. The causes of dry eye syndrome include decreased tear production from lacrimal gland inflammation, decreased oil production from the natural glands in the eyelids (known as meibomian glands), and decreased mucus production from surface goblet cells. Blepharitis is a condition due to inflammation of the mebomian glands that decreases natural oil production. Chalazia or styes are caused by full or partial blockage of these same glands. Warm compresses open the pores of the meibomian glands and restores the normal flow of natural oils to the eye surface; these oils moisturize the eye by preventing evaporation of tears from the eye surface.

In a standardized health survey of 39,876 women in 2003, the incidence of dry eye syndrome in US women was found to be 53-9.8%, affecting an estimated 3.2 million women age 40 and older (1). The prevalence of dry eye syndrome increases with age. The same group estimated 1.68 million men age 50 and over are affected by dry eye syndrome (2),

An industry sponsored survey of 5000 US adults found that 32% reported experiencing, at least one symptom of blepharitis over half the time in the prior 12 months. The same group surveyed 204 US ophthalmologists and optometrists found high rates of blepharitis diagnosed in the outpatient office setting (37% and 47% respectively) (3). The same study found that 4.5% of surveyed adults developed “bumps” on their eyelids (chalazia) which equates to 14 million people (based upon current 2013 US census of 317 million Americans) (3).

The epidemiology of chalazia in US children has not been reported but it known to be a common presenting problem in pediatric eye clinics. Published data from New Delhi, India, showed 12% of 5,012 pediatric patients presenting to the clinic had blepharitis. Of these 615 patients, 18% had chalazia, making blepharitis and chalazia the most common presenting eye problem in the study population (4).

### Currently Available Treatments

In the survey reported by Lemp and Nichols, 69% of patients with blepharitis are prescribed warm compresses as first line intervention (3). Warm compresses are also the first line of therapy for treatment of chalazia, and are frequently recommended for patients with dry eye syndrome as an adjunct therapy to tear replenishment with artificial tears or treatment with pharmaceuticals.

When warm compresses are typically prescribed for patients, physicians advise a regimen of using a face towel, soaked in warm tap water, placed over each eye for 5-20 minutes, to be performed 2-4 times daily for a period of 1-2 months. Therapy may be needed for months, years or indefinitely. Patients often report great difficulty with each step of this prescribed regimen. The face towels often lose heat over a period of 1-3 minutes, necessitating repeated re-warming with tap water. The eye must also he covered sometimes for 20 minutes, daring which time the patient often is unable to do other activities as he or she must hold the towel on the eye and re-warm it frequently. This must he done multiple times daily. Because of these limitations, compliance is frequently suboptimal. Many patients simply give up on treatment after a few short weeks, and subsequently fail to see long term benefits.

Aside from the typically prescribed face towel, other anecdotal means of doing warm compresses include using a warm tea bag, baked potato, commercially available cosmetic facial masks filled with herbs such as flaxseed or wheat husks that can must be microwaved prior to use. A novel device was patented in 2012 called Eye-Press. It utilizes a similar idea of a self-warming device (sodium acetate), but the heat lasts only 3-5 minutes, it must be held to the face by hand and must be boiled to be reused.

### BRIEF SUMMARY OF THE INVENTION

This invention is of a self-warming, hands free compress that allows for continuous application of moist heat to the outer eyelid surface. The compress remains heated for up to 6 hours and may be used multiple times daily.

### BRIEF DESCRIPTION OF THE SEVERAL VIEWS OF THE DRAWING

FIG. 1 (FIG. 1) shows the prototype viewed from the front. The outer mesh layer is external, and the water proof and terry cloth layers are sewn together to form the inner layer. Between the two layers there is a pocket for the warming device. An elastic band secures the device to the head.

FIG. 2 (FIG. 2) shows the prototype viewed from the top. The pocket is demonstrated and labeled in this view.

FIG. 3 (FIG. 3) shows the prototype worn on a model to demonstrate the position of the device on the face.

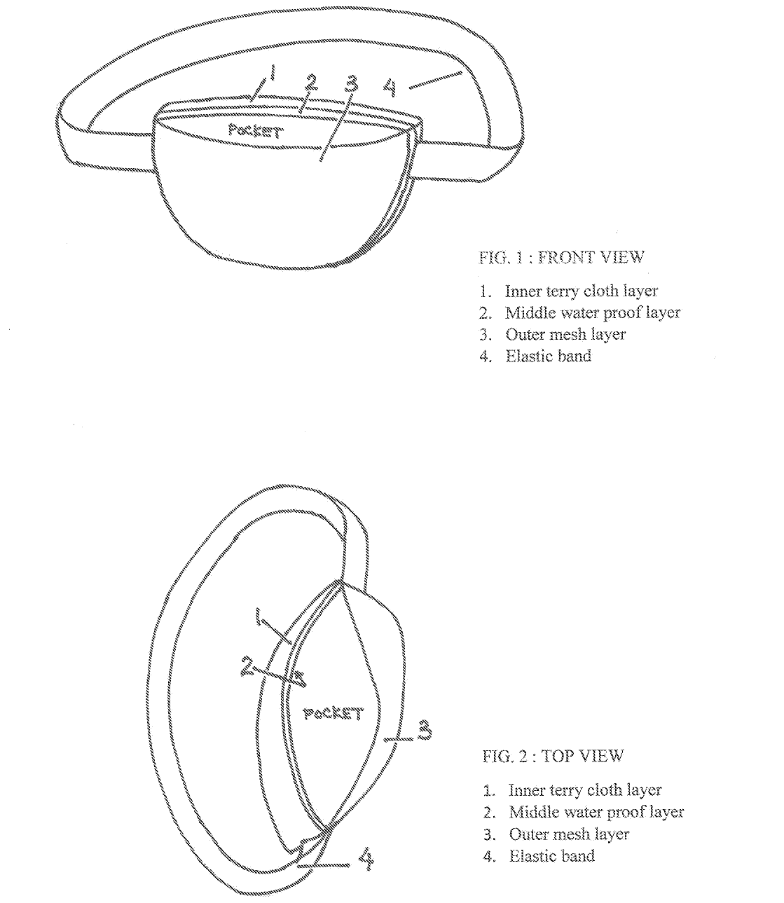

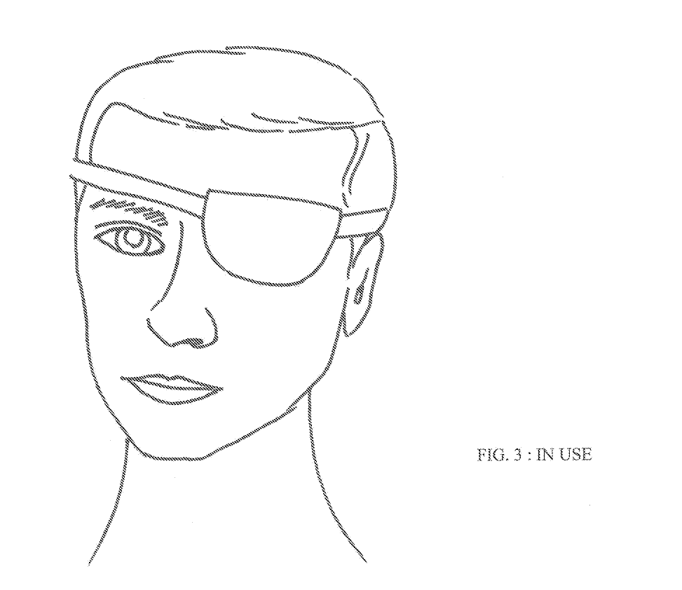

### DETAILED DESCRIPTION OF THE INVENTION

This invention is of a single eye patch made with commercially available fabric that is on one side terry cloth and the other side a water impermeable lining. The terry cloth side is moistened at the time of use with 1 cc of tap water and applied to the outer eyelids. The water impermeable lining separates the wet terry cloth surface from the dry self-heating packet, while still allowing sufficient heat to transfer through the wet terry cloth to the patient's eyelid surface, This creates the critical moist heat environment on the eyelid surface. The terry cloth and water impermeable fabric layer is then stitched to a thin, mesh-like fabric, creating a pocket in which to place the self-heating packet. The mesh-like fabric allows for air flow into the self-heating packet, another critical consideration, as the packet will only produce heat when kept dry and exposed to air. The entire fabric-mesh pocket is attached to an adjustable elastic band that fits comfortably around the patient's head to allow hands-free use.

Self-heating packet technology has been in commercial use for decades and is readily available in the form of self-heating packets for medical and non-medical applications including neck and back warming for relief from arthritic or athletic pains, and hand/toe warming for comfort in cold weather conditions. These packets consist of non-toxic, environmentally friendly iron particles packaged without exposure to oxygen. When these iron particles are exposed to room air (which contains 21% oxygen), they undergo immediate rust formation, which is a heat-generating (exothermic), not vapor forming, chemical reaction. The packets self-heat to an average of 135 degrees Fahrenheit within an average of 7-15 minutes. The ingredients within the packet must be continuously exposed to air and also remain completely dry in order for the heat-generating reaction to continue.

These self-heating packets have been commercially marketed for decades and are safe when applied to the skin. Their usual temperature of 110-120 degrees Fahrenheit is too low to cause skin burns or damage (5). Likewise the eye is not known to be adversely affected by application of heat at this temperature, and there is no known or theoretical damage that may occur with such application of these self-heating packets when used as directed.

The terry cloth and water impermeable fabric is also commercially available as water-proof linings used in baby bibs, cloth diapers, bed mattress covers. Application of heat at 135 degrees Fahrenheit, which is the typical temperature of a clothes dryer on medium/high heat, does not cause degradation or toxic change in the lining (6)

### REFERENCES

1. Schaumberg D A, Sullivan D A, Buring J E, Dana M R. Prevalence of dry eye syndrome among US women. Am J Ophthalmol, 2003 August:136(2):318-26.
2. Schaumberg D A, et al. Prevalence of dry eye disease among US men: estimates from the Physicians' Health Studies, Arch Ophthalmol, 2009;127:763-8.
3. Lemp M A, Nichols K K. Blepharitis in the United States 2009:a survey-based perspective on prevalence and treatment. Ocular Surf. 2009:7(suppl 2):S1-S14
4. Gupta N, Dhawan A. Beni S. D'souza P. Clinical spectrum of pediatric blepharokeratoconjunctivitis. J AAPOS, 2010 December: 14(6):527-9.
5. http://www.uptodate.com/contentsiskin-bums-beyond-the-basics
6. http://www.geappliances.com/search/fast/infobase/10000971.htm

1. The novel aspects of this invention that make it unique include the ability to generate long lasting, self-perpetuating moist heat, with hands free convenience. The only other self heating device on the market similar to this one is the Eye-Press device which is able to remain warm for 3-5 minutes, much less than the commonly prescribed time of 5-20 minutes. Eye-Press must also be boiled to be reused which can be inconvenient for users. These were frequent complaints listed about Eye-Press on amazon.com reviews. Other devices must be microwaved to be warmed. Towels are ineffective due to their limited ability to retain heat for more than 3 minutes they need to be constantly reheated with tap water or microwave. Tea bags, baked potatoes and other home remedies may contain undesirable infectious bacteria or fungi and can be considered not hygienic. No other known therapy allows patients these multiple conveniences.
